(19) United States
(12) Patent Application Publication
(10) Pub. No.: US 2026/0060714 A1
(43) Pub. Date: Mar. 5, 2026

(54) Precision Needle Rotation Mechanism for MRI Guided Procedures

(71) Applicant: Georgia Tech Research Corporation, Atlanta, GA (US)

(72) Inventors: Alexey Khotimsky, Atlanta, GA (US); Daniel Enrique Martinez, Atlanta, GA (US); Waiman Meinhold, Atlanta, GA (US); Jun Ueda, Atlanta, GA (US)

(21) Appl. No.: 19/179,548

(22) Filed: Apr. 15, 2025

Related U.S. Application Data
(60) Provisional application No. 63/634,062, filed on Apr. 15, 2024.

(51) Int. Cl. A61B 34/20; A61B 17/00; A61B 17/34

(52) U.S. Cl. CPC A61B 34/20; A61B 17/3403; A61B 2017/00911; A61B 2017/3409; A61B 2034/2051

(57) ABSTRACT

An exemplary embodiment of the present disclosure provides a system for guiding a probe during a medical procedure. The system can comprise a joint, a shaft, and a motor. The joint can comprise an aperture extending therethrough. The shaft can extend through the aperture and be configured to pivot between a plurality of angles with respect to the joint. The motor can be coupled to the joint and configured to cause a rotation of a first portion of the joint, such that the rotation of the first portion of the joint causes an equal rotation of the shaft, independent of an angle of the shaft with respect to the joint in the plurality of angles.

### CROSS-REFERENCE TO RELATED APPLICATIONS

This application claims the benefit of U.S. Provisional Application Ser. No. 63/634,062, filed on 15 Apr. 2024, which is incorporated herein by reference in its entirety as if fully set forth below.

### FIELD OF THE DISCLOSURE

The various embodiments of the present disclosure relate generally to medical devices, and more particularly to devices for controlling precise needle rotation during MRI guided procedures.

### BACKGROUND

Lower back pain is a common ailment cited in many patients, particularly elderly, with more than 80% of adults expected to be effected at some point in their lifetime. Furthermore, it is responsible for a large amount of economic and personal strain, due to resource utilization and loss in productivity. Radiofrequency ablation (RFA), which is a common procedure to remedy lower back pain through thermal ablation of the medial branch nerve, has been shown to be effective at mitigating symptoms. However, conventional manual methods require manual readjustment of the ablation probe when the trajectory is inaccurate, increasing procedure time, patient discomfort, and decreasing the long-term benefit of RFA.

As an alternative, much work has been done to demonstrate the efficacy of robotic-guided RFA procedures, primarily of tumors. These have shown greater accuracy than their human-guided counterparts, as well as decreased procedure times and decreased radiation exposure to both patient and doctor. To this end, MRI-compatible robots capable of targeting ablation have been widely developed, particularly targeting ablation of tumors in the liver or brain. Though these robots allow for autonomous insertion of the ablation probe, they are often bulkier, table-mounted designs and do not specifically target the lower back region. In order to account for patient motion, Li et al., “Body-mounted robotic assistant for MRI-guided low back pain injection.” International journal of computer assisted radiology and surgery Vol. 15 No. 2 (2020): pp. 321-331, provided a four degree of freedom (DOF) body-mounted parallel plane robot. Meinhold et al., “A direct drive parallel plane piezoelectric needle positioning robot for MRI guided intraspinal injection.” IEEE Transactions on Biomedical Engineering Vol. 68 No. 3 (2020): pp. 807-814, proposed a similar direct-drive parallel plane robot for stem cell injections, accounting for hysteresis, but again leaving probe insertion and rotation to be controlled by a surgeon. There remains a demonstrated lack of a six DOF robot design capable of autonomous insertion and rotation of the probe between MRI-compatible procedures.

RFA probes are often asymmetrical, and the shape of the ablated region thus depends on the orientation of the probe. Furthermore, it has been previously demonstrated that probe rotation in combination with beveled tip geometries can reduce targeting errors by up to 70%. Likewise, this would further increase the accuracy of nerve targeting.

The present disclosure improves upon these conventional designs by providing devices capable of automated control of rotation of the probe about the primary axis of the probe tools independent of probe angle and position. Such embodiments can increase accuracy in procedures such as RFA.

### BRIEF SUMMARY

An exemplary embodiment of the present disclosure provides a system for guiding a probe during a medical procedure. The system can comprise a joint, a shaft, and a motor. The joint can comprise an aperture extending therethrough. The shaft can extend through the aperture and be configured to pivot between a plurality of angles with respect to the joint. The motor can be coupled to the joint and configured to cause a rotation of a first portion of the joint, such that the rotation of the first portion of the joint causes an equal rotation of the shaft, independent of an angle of the shaft with respect to the joint in the plurality of angles.

In any of the embodiments disclosed herein, the joint can be a constant velocity joint.

In any of the embodiments disclosed herein, the constant velocity joint can be a Rzeppa-style constant velocity joint.

In any of the embodiments disclosed herein, the joint can comprise an outer race, and inner race, and a bearing cage. The outer race can be coupled to the motor. The inner race can be coupled to the shaft and can comprise the aperture. The bearing cage can be disposed between the inner and outer races.

In any of the embodiments disclosed herein, the motor can be coupled to the outer race via a belt, such that rotation of the motor induces a rotation of the outer race.

In any of the embodiments disclosed herein, the inner race can be configured to pivot between a plurality of angles with respect to the outer race.

In any of the embodiments disclosed herein, the motor can be a stepper motor configured to rotate among a plurality of steps.

In any of the embodiments disclosed herein, rotation of the motor between adjacent steps can cause an equal rotation of the shaft, independent of the angle of the shaft with respect to the joint in the plurality of angles.

In any of the embodiments disclosed herein, the joint can be made of a magnetic resonance imaging (MRI) compatible material.

In any of the embodiments disclosed herein, the MRI compatible material can be non-magnetic and non-conductive.

In any of the embodiments disclosed herein, the MRI compatible material can comprise polylactic acid (PLA).

In any of the embodiments disclosed herein, the joint can be 3D printed using the MRI compatible material.

In any of the embodiments disclosed herein, the shaft can comprise one of an ablation probe, an injection probe, a biopsy probe, or a lead placement probe.

In any of the embodiments disclosed herein, the probe can be coupled to a distal end of the shaft, and the shaft can be configured such that non-rotational movement of a proximal end of the shaft alters the angle of the shaft with respect to the joint in the plurality of angles.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIGS. 1A-D provide various views of a system for guiding a probe during a medical procedure, in accordance with some embodiments of the present disclosure.

FIG. 2 provides a schematic and image of a continuous velocity joint, in accordance with some embodiments of the present disclosure.

FIG. 3 provides a plot of joint output v. input angle, in accordance with some embodiments of the present disclosure.

FIG. 4 provides a schematic diagram of an experimental setup for a continuous velocity joint validation, in accordance with some embodiments of the present disclosure.

### DETAILED DESCRIPTION

Although preferred exemplary embodiments of the disclosure are explained in detail, it is to be understood that other exemplary embodiments are contemplated. Accordingly, it is not intended that the disclosure is limited in its scope to the details of construction and arrangement of components set forth in the following description or illustrated in the drawings. The disclosure is capable of other exemplary embodiments and of being practiced or carried out in various ways. Also, in describing the preferred exemplary embodiments, specific terminology will be resorted to for the sake of clarity.

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As used in the specification and the appended claims, the singular forms “a,” “an” and “the” include plural referents unless the context clearly dictates otherwise.

Also, in describing the preferred exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges can be expressed herein as from “about” or “approximately” one particular value and/or to “about” or “approximately” another particular value. When such a range is expressed, another exemplary embodiment includes from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, member, particle, or method step is present in the composition or article or method, but does not exclude the presence of other compounds, materials, particles, method steps, even if the other such compounds, material, particles, method steps have the same function as what is named.

Mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

The materials described as making up the various members of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

Various embodiments of the present disclosure provide systems for guiding probes, e.g., ablation probes, during medical procedures (e.g., RFA). As discussed above, disadvantage of conventional systems is their requirement for the surgeon to manually rotate such probes during ablation procedures. Conventional systems were limited to manual rotation, in part, because conventional systems lacked the ability to automatically control rotation through a variety of angle of the shaft/probe during the procedure. For example, a surgeon might routinely need to adjust the angle of approach of the probe to the tissue to be ablated, i.e., the probe may need to be able to transition between a number of angles with respect to the tissue during the RFA procedure. Conventional systems have been unable to provide any accurate control of rotation, independent of the angle of the probe. To overcome these disadvantages, embodiments of the present disclosure utilize specialized joints, e.g., constant velocity joints, that allow for automated precise control of the rotation of the probe, regardless of the angle of the probe.

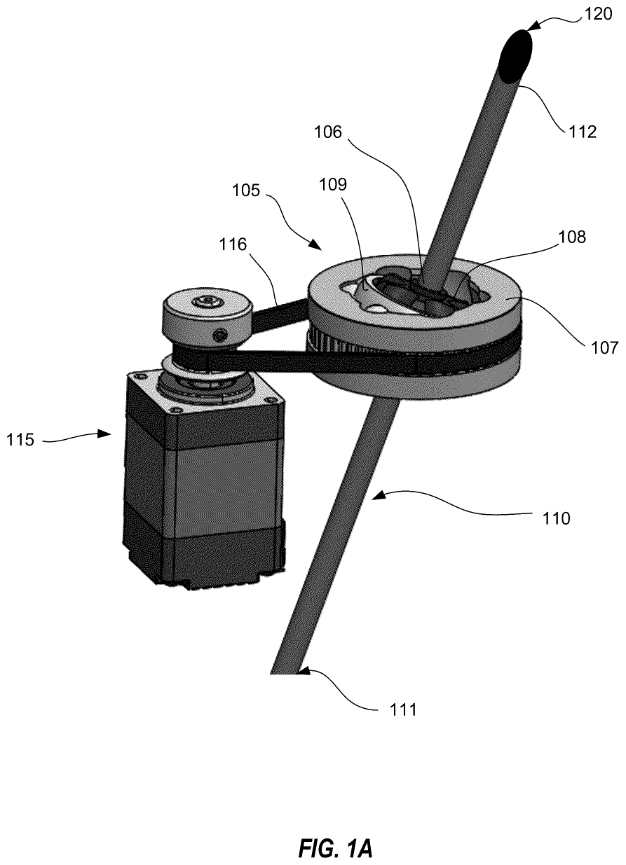

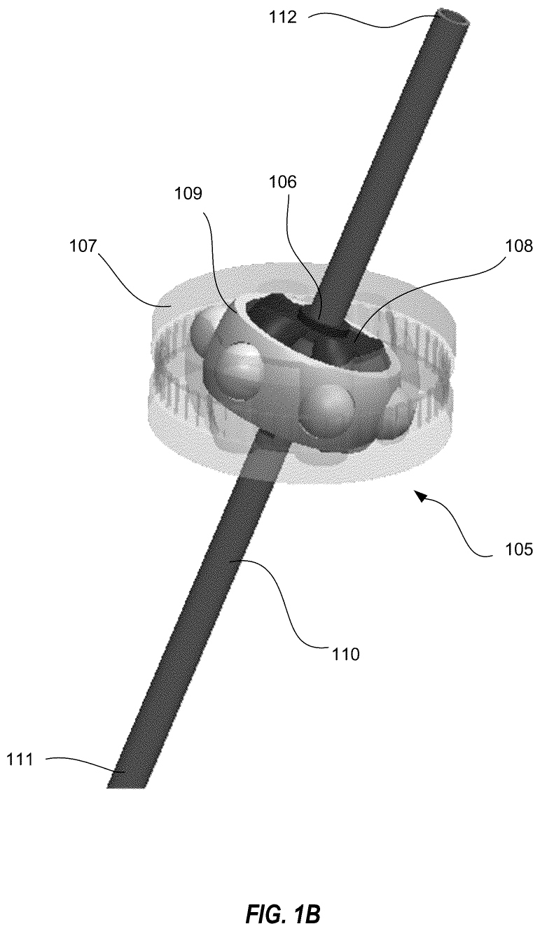

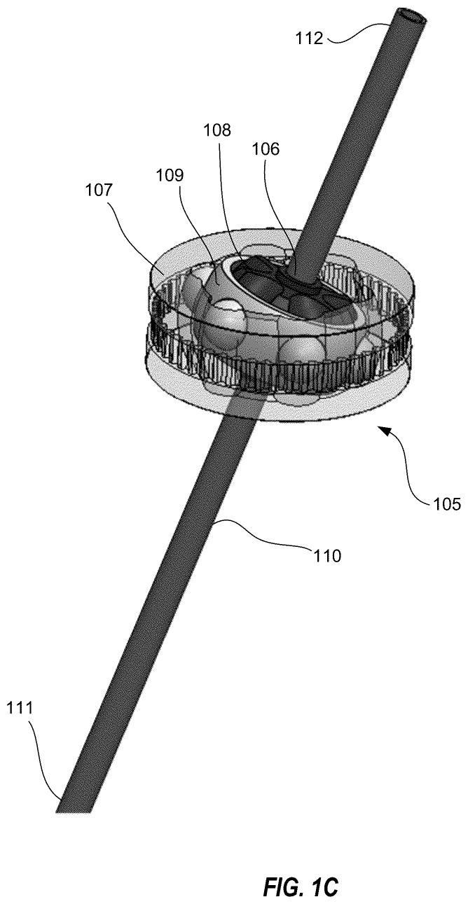

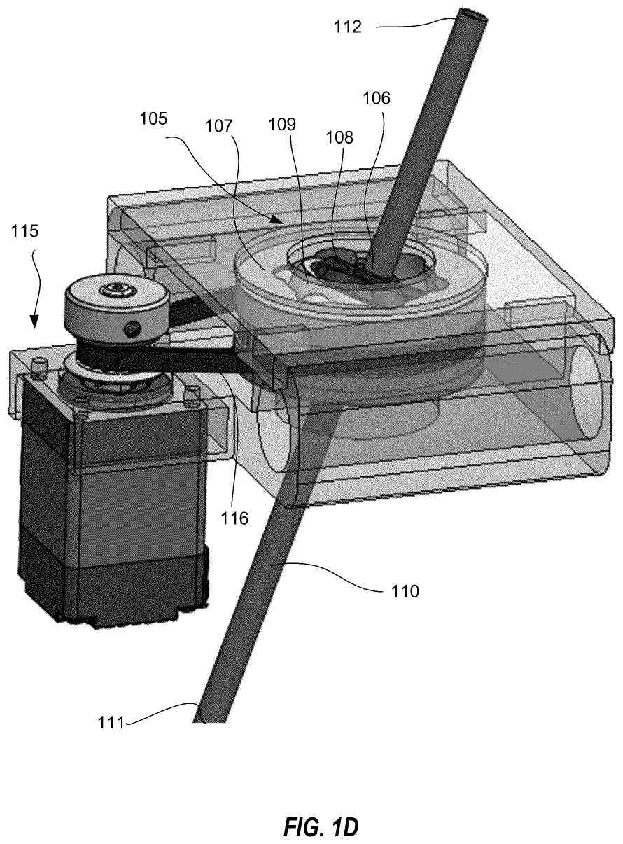

FIGS. 1A-D provide multiple views, with varying degrees of transparency, of exemplary systems of the present disclosure. As shown in FIGS. 1A-D, an exemplary embodiment of the present disclosure provides a system for guiding a probe during a medical procedure. The system can comprise a joint 105, a shaft 110, and a motor 115. The joint 105 can comprise an aperture 106 extending therethrough. The shaft 110 can extend through the aperture 106 and be configured to pivot between a plurality of angles with respect to the joint 105. The distal end 112 of the shaft 110 can comprise a probe 120 for performing a medical procedure. The probe can be many different probes in accordance with various embodiments of the present disclosure, including, but not limited to, an ablation probe, an injection probe, a biopsy probe, a lead placement probe, and the like.

Though not shown in FIGS. 1A-D, the proximal end 111 of the shaft 110 can be coupled to an actuator for controlling the relative X-Y position of the distal end 112 to the joint 105. As would be appreciated by those skilled in the art, for example, if the X-Y position of the joint 105 remains constant and the X-Y position of the proximal end 111 is altered (i.e., non-rotational movement of the proximal end), the angle of the shaft 110 with respect to the joint 105 changes, thereby altering a corresponding X-Y position of the distal end 112 (including the probe 120).

The motor 115 can be coupled to the joint 105 and configured to cause a rotation of a first portion of the joint 105. The joint 105 can be configured such that the rotation of the first portion of the joint causes an equal rotation of the shaft 110, independent of the angle of the shaft 110 with respect to the joint 105. The joint 105 can be many different joints 105 that allow for the coupled rotation described herein.

In some embodiments, the joint 105 can be a constant velocity joint. As used herein, the term “constant velocity joint” generally refers to a mechanical coupling that allows for the transmission of rotational power through a variable angle, at a constant rotational speed, without an appreciable increase in friction or play. This type of joint is designed to maintain a consistent velocity of the output shaft regardless of the angle of the input portion. Many different constant velocity joints are contemplated by the present disclosure, including, but not limited to, a Rzeppa-style constant velocity joint.

FIGS. 1A-D illustrate the use of a Rzeppa-style constant velocity joint 105, in accordance with various embodiments of the present disclosure. As seen in the figures, the joint 105 can comprise an outer race 107, and inner race 108, and a bearing cage 109. The bearing cage 109 can allow multiple bearings to interface with the outer 107 and inner 108 races, such that rotation of outer race 107 results in an equal rotation of the inner race 108. The outer race 108 can be coupled to the motor 115, such as via a belt 116. Thus, actuation of the motor 115 results in a movement of the belt 116 that induces rotation of the outer race 107. The inner race 108 can comprise an aperture 106 extending therethrough and configured to hold the shaft 110 extending through the aperture 106. The bearing cage 109 can be disposed between the inner 107 and outer 108 races and secure bearings.

The shaft 110 can be coupled to the inner race 108 such that the angle of the inner race 108 to the shaft 110 remains substantially constant. The inner race 108, however, can pivot between a plurality of angles with respect to the outer race 107, thus altering the angle of the shaft 110 relative to the joint 105, i.e., the outer race 107 of the joint 105 and thus the tissue to be ablated.

In some embodiments, the system can be configured for use with a magnetic resonance imaging (MRI) machine to guide the surgeon during the medical procedure. Accordingly, in some embodiments, the joint 105 (and other components of the system) can be made of one or more MRI compatible materials. As used herein, the term “MRI compatible material” refers to any material that can be safely used within the environment of a Magnetic Resonance Imaging (MRI) machine without causing interference, artifacts, or safety hazards. For example, exemplary MRI compatible materials can be both non-magnetic and non-conductive. Further, in some embodiments, the MRI compatible material can also be compatible with many additive manufacturing methods (e.g., 3D printing). Such materials, including, but are not limited to polylactic acid (PLA), acrylonitrile butadiene styrene (ABS), polyethylene terephthalate glycol (PETG), polyamide (Nylon), polyether ether ketone (PEEK), thermoplastic polyurethane (TPU), polycarbonate (PC), silicone, various resins, and the like.

The motor 115 used to induce rotation in the joint/shaft/probe can be many different motors, including, but not limited to a stepper motor configured to rotate among a plurality of steps. Such steps can be equally spaced apart, such that rotation of the motor 115 between adjacent steps can cause an equal rotation of the shaft 110, independent of the angle of the shaft 110 with respect to the joint 105.

### Examples

The below example section describes an exemplary system for guiding a probe during a medical procedure. This example is provided for illustrative purposes only and should not be construed as limiting the scope of the present disclosure or the claims appended hereto.

### Materials and Methods

### System Design

The exemplary system comprises two planes modeled off of the spinal injection robot proposed in Meinhold et al. Similarly, the system was built from non-magnetic materials that pose no obstruction to the robot's use inside of MRI. Though not installed for while the CV joint was being validated, the two stages are actuated by piezoelectric linear actuators (MicroMo, Clearwater, FL) have a range of 80 mm. The two ball joints control the position and the angle of a hollow guide, allowing for four DOF control of an ablation probe.

### CV Joint

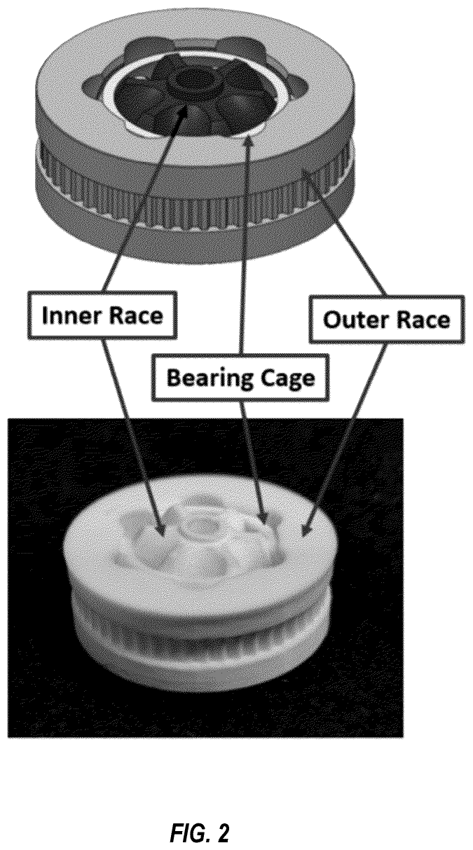

The top ball joint can comprise a novel Rzeppa-style homokinetic joint and can be controlled by a 20 mm×30 mm NEMA 8-size stepper motor (Pololu, Las Vegas, NV) to allow for the needed rotation of an ablation probe. Though this stepper motor is not compatible with an MRI environment, it was chosen as a low-cost alternative to validate the rotation of the CV joint, which can be tested in a magnet-safe environment outside of MRI. The joint was printed out of PLA on an Ender 3 Pro 3D Printer (Ender, Shenzen, China), a low cost and suitable material for use in MRI. It comprises three primary parts, an outer race, an inner race, and a bearing cage, along with ball bearings to transmit rotation regardless of the orientation of the input and output axes. With the outer race of the CV joint having 54 teeth and the pulley having 15 teeth, the gear ratio between the motor and the CV joint is 1:3.6. This results in an output resolution of 0.5° per motor step, given the motor's resolution of 200 steps per revolution. The hollow guide is press-fit into the inner race of the joint and does not slide relative to the direction of the needle and prevents slip. A model and photo of the CV Joint is shown in FIG. 2.

### Materials and MRI Visibility

Though the validation of the CV joint does not require use inside of MRI, the materials selected for the manufacturing of the joint, and the remaining platform are suitable for such environments. The planes of the robot are machined out of acetal resin. The remaining com ponents, such as those with complex geometries, are 3D-printed with polylactic acid (PLA) filament. The hollow guide is a 4 mm brass rod. The fasteners on the robot are nylon and aluminum. To allow for image-guided control of the robot, both stages contain toroidal cavities that are created concentric with the ball joint, which allow for them to be filled with a contrast agent, allowing for imaging inside of MRI, with the rest of the robot not being visible.

### Experimental Setup

### Validating Homokinetic Behavior

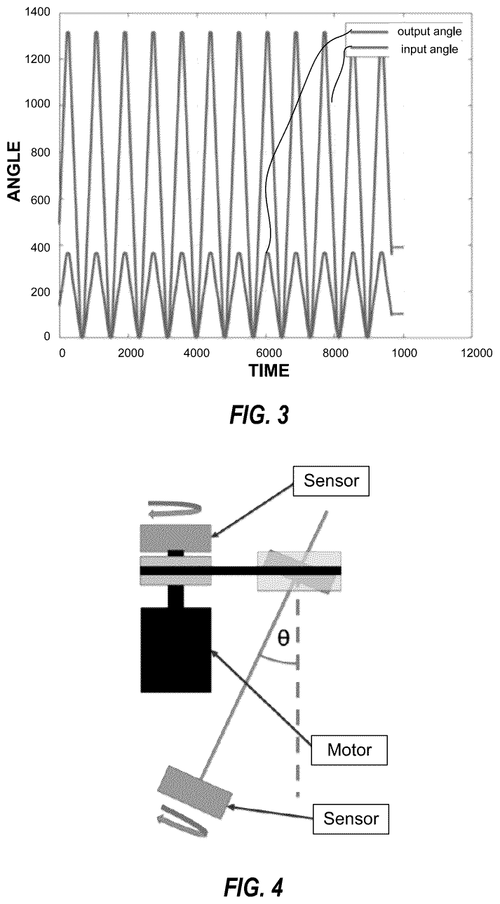

The open-loop repeatability of the CV joint was measured with a Polhemus FASTRAK (Polhemus, Colchester, VT) six DOF motion capture system. Localizers were attached in-line with the axes of both the input motor to the CV joint and the output probe. The input motor to the CV joint was actuated 720 steps in one direction, and then 720 steps in reverse to return to its original position, corresponding to a complete rotation of the output axis. The diagram for this experimental setup is shown in FIG. 4. This actuation was then performed ten times in a row, and sample resulting measurements from the input and output angles are shown in FIG. 3. This procedure was then repeated for various configurations of the robot, with 0, as shown in FIG. 4 ranging from 0°-35° at intervals of 5°, representing the maximum physical range of the robot.

### Results and Discussion

### Accuracy

It is shown that the CV joint is capable of maintaining a linear relationship between the input and output angles between the motor and the probe, and is almost entirely agnostic to the robot configuration. The relationship between the output and input angle compares to an expected linear relationship with slope 3.6, corresponding to the gear ratio of the system. This demonstrates that it is capable of utilization with elementary kinematics, as the experimental data closely follows the expected slope. The root-mean-square error (RMSE), when measured against the ideal linear relationship, saw a minimum of 2.9244° at an offset angle of 30° and a maximum of 6.0437°. The exact errors over the trials at each offset angle are shown in Table 1. During the first 100° of rotation, there is sinusoidal behavior about the expected linear relationship, which could be caused by any misalignment between the FASTRAK sensor and the probe itself, is identified as a potential source of error, though the effect on the RMSE would be expected to cancel over the entire range of motion. Ultimately, the novel MRI-compatible CV joint is capable of accurately transmitting rotation when implemented in a parallel stage MRI-compatible robot regardless of offset angle between the actuator and a probe that it rotates. Besides the simpler kinematics and reduced footprint that the joint allows, such rotation, when implemented in RFA procedures, has the potential to drive down operating time, costs, and errors. As the joint is currently validated with traditional, non-MRI-safe stepper motors, further work is required to test the closed-loop control of the robot inside the MRI setting, as well as experiments for demonstrating the rotation of the CV joint for lesion targeting in a phantom. As those skilled in the art would appreciate, the accuracy of the CV joint could be further improved by increasing the precision of the manufacturing procedure, such as utilizing stereolithography or selective laser sintering 3D-printing techniques, which could optimize the CV joint tolerance, another likely cause for the little errors that were observed.

TABLE 1
ERROR VS. CONFIGURATION ANGLE

| Configuration | 0 | 5 | 10 | 15 | 20 | 25 | 30 | 35 |
|---|---|---|---|---|---|---|---|---|
| Mean Abs | 3.6965 | 2.8285 | 2.8562 | 3.2604 | 2.7221 | 5.3366 | 2.3483 | 4.8283 |
| Err (°) | | | | | | | | |
| RMSE (°) | 4.3629 | 3.5560 | 3.7299 | 3.9987 | 3.4150 | 6.0437 | 2.9244 | 5.5638 |

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A system for guiding a probe during a medical procedure, the system comprising:
a joint comprising an aperture extending therethrough;
a shaft extending through the aperture and configured to pivot between a plurality of angles with respect to the joint;
a motor coupled to the joint and configured to cause a rotation of a first portion of the joint, such that the rotation of the first portion of the joint causes an equal rotation of the shaft, independent of an angle of the shaft with respect to the joint in the plurality of angles.

2. The system of claim 1, wherein the joint is a constant velocity joint.

3. The system of claim 2, wherein the constant velocity joint is a Rzeppa-style constant velocity joint.

4. The system of claim 3, wherein the joint comprises:
an outer race coupled to the motor;
an inner race coupled to the shaft, wherein the inner race comprises the aperture; and
a bearing cage disposed between the inner and outer races.

5. The system of claim 4, wherein the motor is coupled to the outer race via a belt, such that rotation of the motor induces a rotation of the outer race.

6. The system of claim 4, wherein the inner race is configured to pivot between a plurality of angles with respect to the outer race.

7. The system of claim 1, wherein the motor is a stepper motor configured to rotate among a plurality of steps.

8. The system of claim 7, wherein rotation of the motor between adjacent steps causes an equal rotation of the shaft, independent of the angle of the shaft with respect to the joint in the plurality of angles.

9. The system of claim 1, wherein the joint is made of a magnetic resonance imaging (MRI) compatible material.

10. The system of claim 1, wherein the MRI compatible material is non-magnetic and non-conductive.

11. The system of claim 10, wherein the MRI compatible material comprises polylactic acid (PLA).

12. The system of claim 9, wherein the joint is 3D printed using the MRI compatible material.

13. The system of claim 1, wherein the shaft comprises a probe selected from the group consisting of: an ablation probe, an injection probe; a biopsy probe, and a lead placement probe.

14. The system of claim 13, wherein the probe is coupled to a distal end of the shaft, and wherein the shaft is configured such that non-rotational movement of a proximal end of the shaft alters the angle of the shaft with respect to the joint in the plurality of angles.

15. A system for guiding a probe during a medical procedure, the system comprising:
a constant velocity joint comprising an aperture extending therethrough, wherein the constant velocity joint is made of a magnetic resonance imaging (MRI) compatible material;
a shaft extending through the aperture and configured to pivot between a plurality of angles with respect to the joint, the shaft comprising a probe;
a motor coupled to the joint and configured to cause a rotation of a first portion of the joint, such that the rotation of the first portion of the joint causes an equal rotation of the shaft, independent of an angle of the shaft with respect to the joint in the plurality of angles.

16. The system of claim 15, wherein the constant velocity joint is a Rzeppa-style constant velocity joint, comprising:
an outer race coupled to the motor;
an inner race coupled to the shaft, the inner race comprising the aperture, the inner race configured to pivot between a plurality of angles with respect to the outer race; and
a bearing cage disposed between the inner and outer races.

17. The system of claim 16, wherein the motor is coupled to the outer race via a belt, such that rotation of the motor induces a rotation of the outer race.

18. The system of claim 15, wherein the motor is a stepper motor configured to rotate among a plurality of steps.

19. The system of claim 18, wherein rotation of the motor between adjacent steps causes an equal rotation of the shaft, independent of the angle of the shaft with respect to the joint in the plurality of angles.

20. The system of claim 15, wherein the probe is coupled to a distal end of the shaft, and wherein the shaft is configured such that non-rotational movement of a proximal end of the shaft alters the angle of the shaft with respect to the joint in the plurality of angles.
